(19) United States
(12) Patent Application Publication
(10) Pub. No.: US 2018/0125462 A1
(43) Pub. Date: May 10, 2018

(54) PROBE FOR ULTRASONIC DIAGNOSTIC APPARATUS

(71) Applicant: Samsung Electronics Co., Ltd., Gyeonggi-do (KR)

(72) Inventors: Chang-yeon WON, Seoul (KR); Chang-min KIM, Seoul (KR); Sang-dong JANG, Gyeonggi-do (KR); Yoon-seok KIM, Gyeonggi-do (KR); Jung-min KIM, Seoul (KR)

(21) Appl. No.: 15/797,037

(22) Filed: Oct. 30, 2017

(30) Foreign Application Priority Data
Nov. 7, 2016 (KR) 10-2016-0147635

(51) Int. Cl. A61B 8/00

(52) U.S. Cl. CPC A61B 8/546; A61B 8/4461; A61B 8/12; A61B 8/4483; A61B 8/4455

(57) ABSTRACT

Provided is a probe for an ultrasonic diagnostic apparatus. The probe includes: the transducer module including a transducer configured to transmit or receive ultrasound waves; a first casing positioned below the transducer module to support the transducer module; a second casing extending along a perimeter of the first casing and integrally formed with the first casing; and a heat spreader positioned below the transducer module and above the first casing, where the heat spreader is a sheet of metal.

### CLAIM OF PRIORITY

This application claims the benefit of Korean Patent Application No. 10-2016-0147635, filed on Nov. 7, 2016, in the Korean Intellectual Property Office, the disclosure of which is incorporated herein in its entirety by reference.

### BACKGROUND

### 1. Field

The present disclosure generally relates to probes for ultrasonic diagnostic apparatuses capable of obtaining ultrasound images for diagnosis of diseases in subjects such as humans or animals.

### 2. Description of the Related Art

In general, ultrasonic diagnostic apparatuses are configured to obtain images with respect to single layers of soft tissue or blood flow. These images are obtained by transmitting ultrasound waves from the surface of the subject to the desired interior portion of the subject, and then receiving the ultrasound waves reflected from the interior portion.

A typical ultrasonic diagnostic apparatus may include a main body, a probe configured to transmit ultrasound signals to the subject and receive signals reflected from the subject, a display unit provided at the upper part of the main body to display the images generated from the received ultrasound signals, and a control panel, typically adjacent to the display unit, for allowing the user to operate the ultrasonic diagnostic apparatus.

In more detail, the probe typically includes a transducer module that, during its operation, may generate a significant amount of heat. To prevent the probe from overheating, the probe may be further equipped with a heat sink and a heat transfer member to dissipate the heat generated by the transducer module. But including known heat sinks and heat transfer members may significantly increase the structural complexity of the probe, thereby causing difficulties when designing the probe. Also, inclusion of known heat sinks and heat transfer members also increases the overall size and weight of the probe, which may be undesirable. Accordingly, in the prior art, manufacture and design of ultrasonic probes present significant challenges.

### SUMMARY

Provided are probes for ultrasonic diagnostic apparatuses capable of obtaining ultrasound images for diagnosis of diseases of a subject.

Additional aspects of the present disclosure will be set forth in part in the description which follows and, in part, will be apparent from the description, or may be learned by practice of the presented embodiments.

According to an aspect of an embodiment, a probe for an ultrasonic diagnostic apparatus includes: a transducer module including a transducer configured to transmit or receive ultrasound waves; a first casing positioned below the transducer module to support the transducer module; a second casing extending along a perimeter of the first casing and integrally formed with the first casing; and a heat spreader positioned below the transducer module and above the first casing, the heat spreader being a sheet of metal.

The first casing and the second casing may each be made of at least one material selected from polyphenylene sulfide (PPS), polyamide66+glass fiber (PA66+GF), polyphthalamide+GF (PPA+GF), terephthalic acid+GF (TPA+GF), polyetheretherketone+GF (PEEK+GF), PA66+long carbon fiber (PA66+LCF), PA66+long-glass fiber reinforced (PA66+LGF), polycarbonate+GF (PC+GF), PC+CF, PEEK, polyphenylsulfone (PPSU), polysulfone (PSU), PPS+GF+melamine formaldehyde resin_{—} (PPS+GF+MF), nylon-6+elastomer, and nylon-6+elastomer+GF.

The first and second casings may be integrally injection molded.

The heat spreader may have a thickness of 3 mm to 10 mm.

The transducer module further may include an acoustic reflective layer provided at a rear surface of the transducer to reflect ultrasound waves transmitted to a rear of the transducer and a backing layer provided at a rear surface of the acoustic reflective layer to prevent ultrasound waves from being transmitted to the rear of the transducer.

The probe may further include a third casing fitted to the second casing and in a shape of a handle adapted to fit a user's hand.

The probe may further include a heat sink in contact with the heat spreader and configured to receive heat from the heat spreader.

The heat sink may include at least one heat sink plate having a planar shape.

The first casing may include at least one heat sink through hole, such that a portion of the at least one heat sink plate passes through the at least one heat sink through hole.

The at least one heat sink plate may be made of graphite.

The probe may further include a conductive oil accommodated in the second casing and having a higher thermal conductivity than air.

The conductive oil may be in contact with a portion of the heat sink and may be configured to transfer heat from the transducer to the heat sink.

The probe may further include a printed circuit board (PCB) electrically connected to the transducer and a support frame configured to support the PCB, where the support frame is integrally formed with the first casing.

The first casing, the second casing, and the support frame may be integrally injection molded with one another.

### BRIEF DESCRIPTION OF THE DRAWINGS

These and/or other aspects will become apparent and more readily appreciated from the following description of the embodiments, taken in conjunction with the accompanying drawings, in which reference numerals denote structural elements:

FIG. 1 is a perspective view of a probe for an ultrasonic diagnostic apparatus according to an embodiment;

FIG. 2 is an exploded perspective view of the probe of FIG. 1;

FIG. 3 is a schematic cross-sectional view of a transducer module according to an embodiment;

FIG. 4A is a perspective view of an upper casing of a probe according to an embodiment;

FIG. 4B is a cross-sectional view of the upper casing taken along a line A-A′ of FIG. 4A;

FIG. 4C is a cross-sectional view of the upper casing taken along a line B-B′ of FIG. 4A; and

FIG. 5 is a schematic front view of a probe for an ultrasonic diagnostic apparatus according to another embodiment.

### DETAILED DESCRIPTION

The present specification describes principles of the present disclosure, sets forth embodiments thereof to clarify the scope of the present disclosure, and may allow those of ordinary skill in the art to implement the embodiments. Additional undisclosed embodiments are possible and the disclosed embodiments should not be construed to limit the spirit of the present disclosure.

Like reference numerals refer to like elements throughout the present specification. The present specification does not describe all components in the embodiments, and therefore common knowledge in the art will be omitted below. Also, when two embodiments include like elements, and the element is already described for one embodiment, description of the corresponding element in the second embodiment may be omitted. The terms “part” and “portion” are not limited to their single or plural forms. Thus, a plurality of “parts” or “portions” may form a single unit or element, or one “part” or “portion” may include a plurality of units or elements.

Furthermore, in the present specification, the “subject” may be the target to be imaged, which may be a human, an animal, or a part of a human or animal. For example, the subject may be a body part, such as an organ, or a phantom.

Throughout the specification, an “ultrasound image” refers to the image of the subject which is generated from ultrasound signals transmitted to and reflected from the subject.

Embodiments will now be described more fully hereinafter with reference to the accompanying drawings.

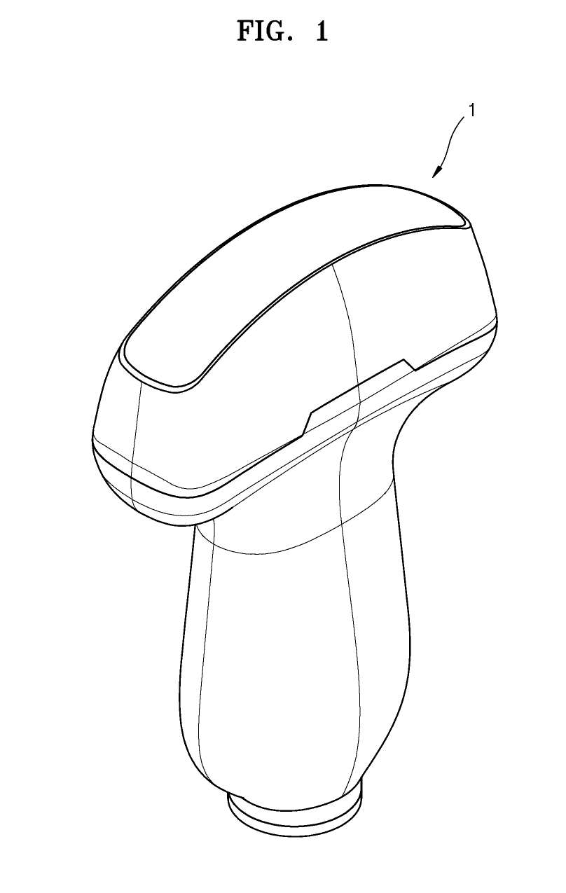

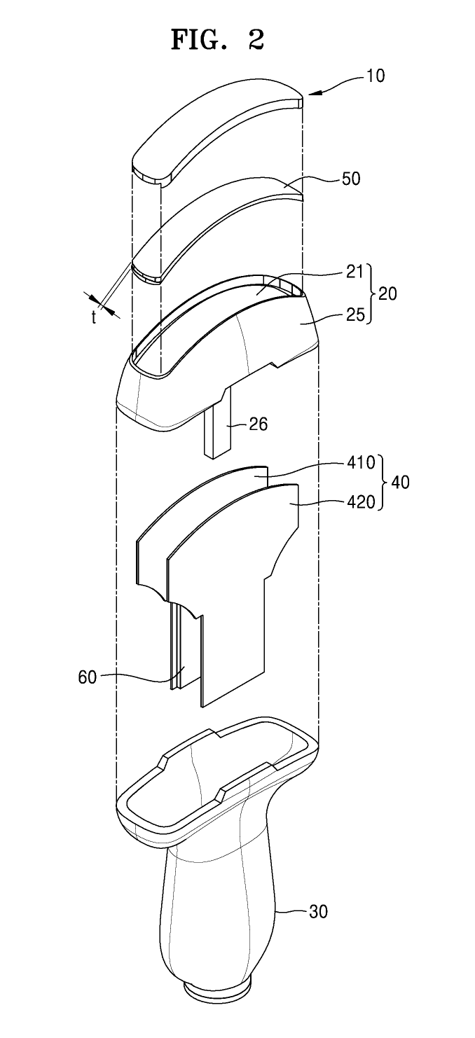

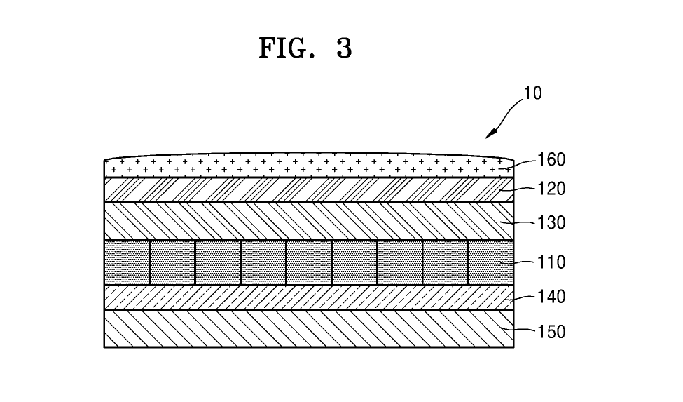

FIG. 1 is a perspective view of a probe 1 for an ultrasonic diagnostic apparatus according to an embodiment, and FIG. 2 is an exploded perspective view of the probe of FIG. 1. FIG. 3 is a schematic cross-sectional view of a transducer module 10 included in the probe 1, according to an embodiment.

Referring to FIGS. 1 through 3, the probe 1 according to the present embodiment is a diagnostic device configured to transmit ultrasound signals to the subject being examined and receive signals reflected from the subject. The probe 1 may include a transducer module 10. The transducer module 10 in turn may include a transducer 110 for transmitting and receiving ultrasound signals. The probe 1 may further include an upper casing 20 for supporting the transducer module 10, a third casing 30 in the shape of a handle to allow the user to hold the probe 1, a heat sink 40 for dissipating heat generated by the transducer module 10, a heat spreader 50 for transferring heat generated by the transducer module 10 to the heat sink 40, and a printed circuit board (PCB) 60 electrically coupled to the transducer 110.

The transducer module 10 may include the transducer 110, a plurality of matching layers 120 and 130 provided in front of the transducer 110 (i.e. between the transducer 110 and the subject), an acoustic reflective layer 140, and a backing layer 150 sequentially positioned behind the transducer 110.

According to an embodiment, the transducer 110 converts electrical energy it receives into ultrasound waves and transmits the ultrasound waves to the subject, i.e. in a forward direction towards the plurality of matching layers 120 and 130. When these ultrasonic waves are reflected by the body of the subject, the transducer 110 receives them and converts them into electrical energy. To this end, the transducer 110 may be a magnetostrictive ultrasonic transducer that utilizes magnetostrictive effects exhibited by magnetic materials, or a piezoelectric ultrasonic transducer that utilizes piezoelectric effects exhibited by piezoelectric materials. The transducer 110 may also be a capacitive micromachined ultrasonic transducer (cMUT) that utilizes vibrations of several hundreds or thousands of micromachined thin films to transmit or receive ultrasound waves. For simplicity of explanation, it is assumed hereinafter that the transducer 110 is a piezoelectric ultrasonic transducer. However, as explained above, the transducer 110 is not so limited.

The matching layers 120 and 130 may be provided between the transducer 110 and the subject. The matching layers 120 and 130 are used to match the acoustic impedances between the transducer 110 and the subject so that ultrasound signals generated by the transducer 110 may be efficiently transmitted to the subject. For this purpose, each of the matching layers 120 and 130 may be made of a material whose acoustic impedance is between those of the transducer 110 and the subject. Suitable materials may include glass or resin. In one example, each of the matching layers 120 and 130 may be made of different materials having different acoustic impedances. For example, the acoustic impedance of the matching layer 120 may be closer to that of the subject, and the acoustic impedance of the matching layer 130 may be closer to that of the transducer 110. In this case, it is possible to gradually reduce the difference between acoustic impedances of the transducer 110 and the subject.

The acoustic reflective layer 140 may be positioned to the rear of the transducer 110 to amplify the vibrational energy generated by the transducer 110 in the direction towards the subject. For example, some of the ultrasound waves generated by the transducer 110 may be initially transmitted towards the acoustic reflective layer 140, i.e. away from the subject. The acoustic reflective layer 140 reflects these ultrasound waves back towards the subject, thereby increasing the acoustic pressure amplitude of the ultrasound waves transmitted from the transducer 110 towards the subject. To this end, the acoustic reflective layer 140 may be made of a material with an acoustic impedance higher than that of the transducer 110.

The backing layer 150 may be positioned to the rear of the acoustic reflective layer 140 to fix the transducer 110 in place and suppress free vibrations of the transducer 110. Because the transducer 110 is prevented from excessively vibrating, the backing layer 150 may also reduce the pulse width of the ultrasound waves generated by the transducer 110. The backing layer 150 also blocks unnecessary propagation of ultrasound waves to the rear of the transducer 110. These propagations to the rear of the transducer 110 may be undesirable because they may cause distortions in the images of the subject. For example, the backing layer 150 is provided in plural and depending on the design, total acoustic impedance of the backing layer 150 may be combined in various ways, e.g., by making one acoustic impedance equal to or higher than another. Thus, it is possible to easily obtain combinations of acoustic impedances that permit absorption or reflection of ultrasound waves

An acoustic lens 160 is positioned in front of the matching layers 120 and 130. The acoustic lens 160 may be used to focus the ultrasound waves on a particular point on the subject. The acoustic lens 160 may have a curved shape to accommodate curved surfaces of the subject.

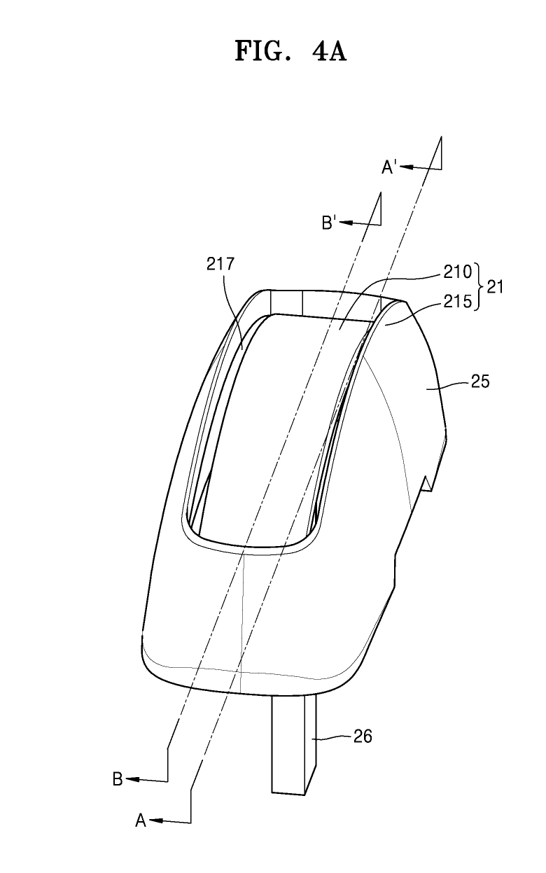

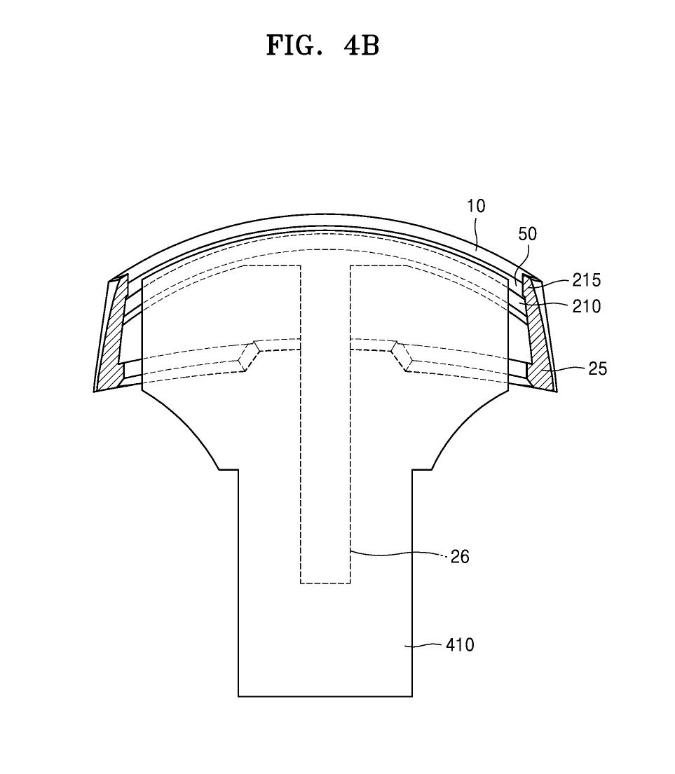

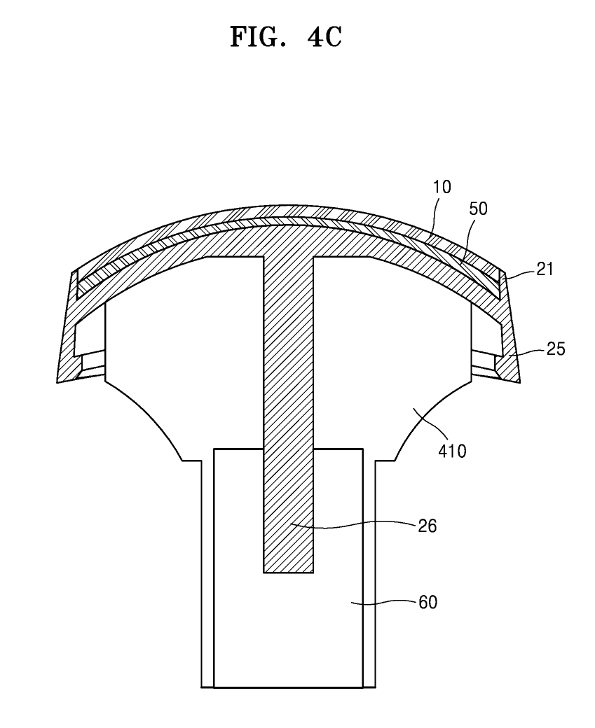

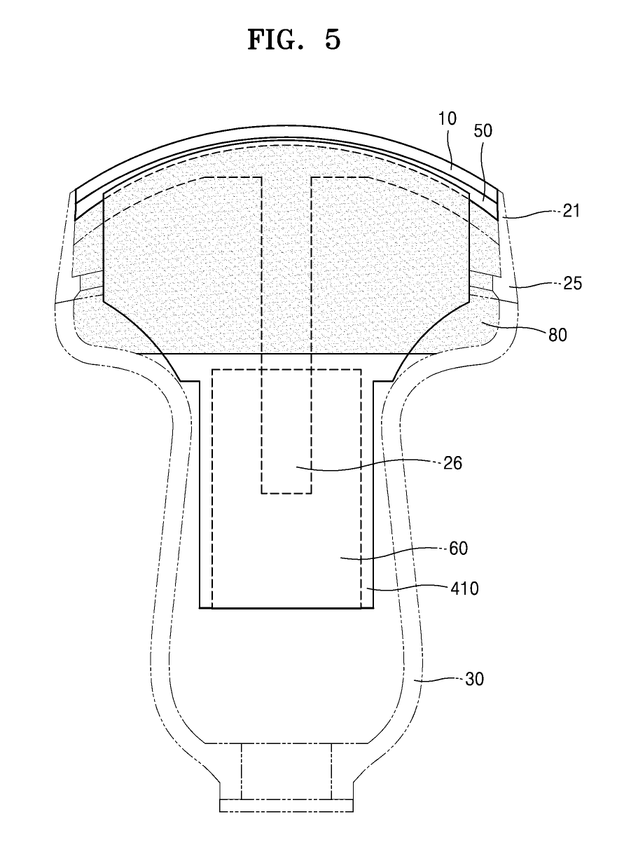

The upper casing 20 may include a first casing 21 for supporting the transducer module 10 and a second casing 25 fitted integrally with the first casing 21. For example, to support the transducer module 10, the first casing 21 may have the shape of a plane having a specific curvature that corresponds to the curvature of the transducer module 10. Furthermore, the second casing 25 may extend along the perimeter of the first casing 21. Together, the first and second casings 21 and 25 may enclose the heat spreader 50 and conductive oil 80, which are described in further detail below. According to an embodiment, the first casing 21 and the second casing 25 may be integrally formed with each other, i.e. molded from a single piece of material. For example, the first and second casings 21 and 25 may be made of resin materials such as polyphenylene sulfide (PPS), polyamide66+glass fiber (PA66+GF), polyphthalamide+GF (PPA+GF), terephthalic acid+GF (TPA+GF), polyetheretherketone+GF (PEEK+GF), PA66+long carbon fiber (PA66+LCF), PA66+long-glass fiber reinforced (PA66+LGF), polycarbonate+GF (PC+GF), PC+CF, PEEK, polyphenylsulfone (PPSU), polysulfone (PSU), PPS+GF+melamine formaldehyde resin (PPS+GF+MF), nylon-6+elastomer, and nylon-6+elastomer+GF. In this case, the first and second casings 21 and 25 may be formed using injection molding. Arrangement of the upper casing 20, the transducer module 10, and the heat spreader 50 will be described in more detail below with reference to FIGS. 4 and 5.

The third casing 30 may be positioned below the upper casing 20 and be in the shape of a handle adapted to fit the user's hand. According to an embodiment, like the first and second casings 21 and 25, the third casing 30 may be made of resin materials such as PPS, PA66+GF, PPA+GF, TPA+GF, PEEK+GF, PA66+LCF, PA66+LGF, PC+GF, PC+CF, PEEK, PPSU, PSU, PPS+GF+MF, nylon-6+elastomer, and nylon-6+elastomer+GF. In this case, the third casing 30 may be formed using injection molding. The heat sink 40 and the PCB 60 may be housed within the third casing 30, as will be described below.

The heat sink 40 may act as a conduit to direct heat from the heat spreader 50 so that the heat can be radiated to the exterior of the probe 1. For example, the heat sink 40 may include first and second heat sink plates 410 and 420. As shown in FIG. 2, the first and second heat sink plates 410 and 420 may be positioned on either side of the third casing 30 such that the top side of the first and second heat sink plates 410 and 420 contact the heat spreader 50 while the bottom side thereof contact a heat sink (not shown) positioned at the bottom of the third casing 30. The first and second heat sink plates 410 and 420 may be made of graphite, but the embodiments are not so limited.

The heat spreader 50 is a heat transfer unit for transferring heat generated by the transducer module 10 to the heat sink 40. According to an embodiment, the heat spreader 50 may be positioned below the transducer module 10 and above the first casing 21 and have a shape corresponding to that of the transducer module 10 and the first casing 21. To allow it to properly transfer heat, the heat spreader 50 may be a heat conductive layer with a predetermined thickness. For example, the heat spreader 50 may be a sheet of metal which has relatively good thermal conductivity, such as aluminum (Al). In this case, the heat spreader 50 may have a thickness t of 3 mm to 10 mm. However, the above is only an example and the embodiments are not so limited. In other embodiments, another metal layer whose thermal conductivity is higher than air may be used as the heat spreader 50.

The PCB 60 may be disposed in the third casing 30 and be connected to electrodes (not shown) of the transducer 110 using wires. For example, the PCB 60 may be supported by a support frame 26, which may be a component that is integrated with the first casing 21. Furthermore, the PCB 60 may further include a connector (not shown), where the connector may be a flexible PCB (FPCB) that can be configured in various shapes.

FIG. 4A is a perspective view of the upper casing 20 according to an embodiment, FIG. 4B is a cross-sectional view of the upper casing 20 taken along line A-A′ of FIG. 4A, and FIG. 4C is a cross-sectional view of the upper casing 20 taken along line B-B′ of FIG. 4A.

Referring to FIGS. 4A through 4C, the upper casing 20 may include the first casing 21 for supporting the transducer module 10 and the heat spreader 50, the second casing 25 extending along the perimeter of the first casing 21, and the support frame 26 extending downwards from the first casing 21. According to an embodiment, in order to support the transducer module 10 and the heat spreader 50, the first casing 21 may include a base 210 having a planar shape of a specific curvature and a support guide 215 protruding from the base 210so as to form a wall for the base 210. Thus, when the heat spreader 50 is a sheet of metal having the thickness t as described above, the transducer module 10 and the heat spreader 50 may be sequentially stacked on the base 210 and be fixedly supported thereon by the support guide 215.

Furthermore, one or more heat sink through holes 217 may be formed between the base 210 and the support guide 215. As shown in FIG. 4A, the one or more heat sink through holes 217 may be in the shape of slits along the sides of the base 210. Thus, as shown in FIG. 4B, the top of the first heat sink plate 410 may pass through the heat sink through hole 217 and be brought into contact with the heat spreader 50. This way, heat may be transferred from the heat spreader 50 to the first heat sink plate 410. Similarly, the top of the second heat sink plate 420 may be exposed via the heat sink through hole 217, and heat transfer between the second heat sink plate 420 and the heat spreader 50 occurs in substantially the same way as that between the first heat sink plate 410 and the heat spreader 50. Thus, for convenience, detailed descriptions thereof will be omitted.

As mentioned above, the second casing 25 is a housing extending along the perimeter of the first casing 21. For example, the second casing 25 may be an outer wall extending along the perimeter of the first casing 21. As explained above, the second casing 25 may couple with the third casing 30 to provide a housing for accommodating various components of the probe 1. Because the heat spreader 50 is positioned above the first casing 21, i.e. outside the first casing 21, and because the first and second casings 21 and 25 may integrally formed as a single piece via injection molding, the manufacture of the probe 1 may be simplified. Furthermore, this arrangement eliminates the need for separate support structures to support the heat spreader 50, so that the probe 1 can be made to be more compact.

The support frame 26 extending downwards may be attached to the bottom of the first casing 21, and in particular, to the bottom of the base 210. Like the first and second casings 21 and 25, the support frame 26 may be made of resin materials such as PPS, PA66+GF, PPA+GF, TPA+GF, PEEK+GF, PA66+LCF, PA66+LGF, PC+GF, PC+CF, PEEK, PPSU, PSU, PPS+GF+MF, nylon-6+elastomer, and nylon-6+elastomer+GF. In this case, the support frame 26 may be integrally molded with the first and second casings 21 and 25 using injection molding. When the support frame 26 is integrally molded with the first and second casings 21 and 25, the PCB 60 may be directly supported by the support frame 26 as shown in FIG. 4C. Accordingly, manufacture of the probe 1 may be simplified because the support frame 26 is integrated formed with first and second casings 21 and 25 in a single injection molding process. A separate support member for supporting the PCB 60 is not required. And because a separate support member is not required, the probe 1 can be made to be more compact. Furthermore, elimination of a separate support member reduces physical interferences among various components included in the probe 1, thereby enhancing product reliability.

FIG. 5 is a schematic front view of a probe 1 for an ultrasonic diagnostic apparatus according to another embodiment. For convenience, descriptions of substantially the same components as in the above embodiment will be omitted below.

As described above, the heat spreader 50 is a heat transfer unit that transfers heat generated by the transducer module 10 to the heat sink 40. According to an embodiment, the heat spreader 50 may be a sheet of metal. However, heat transfer using metal may not be efficient.

Referring to FIG. 5, the probe 1 according to the present embodiment may further include a conductive oil 80, which may be positioned below the transducer module 10 and accommodated in the second casing 25. For example, the conductive oil 80 may be a fluid having a higher thermal conductivity than air, such as a fluid having a thermal conductivity of 0.128 W/mK, which is about five times (5) higher than that of air and having a electric insulation. The conductive oil 80 may be encased in the second casing 25 such that it comes into contact with the heat sink 40, e.g., a first heat sink plate 410, thereby allowing heat generated in the transducer 110 to be efficiently transferred to the heat sink 40.

Furthermore, since the conductive oil 80 is a fluid, it may be accommodated within the first and second casings 21 and 25 without affecting the positions of other components, such as the support frame 26. For example, even when the support frame 26 is integrally formed with the first casing 21, as shown in FIG. 5, the conductive oil 80 may be accommodated in the first and second casings 21 and 25.

According to the above-disclosed embodiments, the disclosed probe includes simplified and integrally formed structures for mounting heat sinks, heat spreaders, and/or other heat transfer members, such that the disclosed probe is made to be compact and lightweight. Furthermore, by integrally forming various support casings, the manufacturing process of the probe may be simplified.

The above description is provided for illustration, and it will be understood by those of ordinary skill in the art that various changes in form and details may be readily made therein without departing from essential features and the spirit and scope of the present disclosure as defined by the following claims. Accordingly, the above embodiments and all aspects thereof are examples only and are not limiting. For example, components described as separate components may be implemented in an integrated manner. Having thus described different embodiments of ultrasonic probes, it should be apparent to those skilled in the art that certain advantages have been achieved, including improved manufacturing of the probes and more compact and lightweight probes.

The scope of the present disclosure is defined not by the detailed description thereof but by the appended claims, and all features within the scope of the appended claims and their equivalents will be construed as being included in the present disclosure.

What is claimed is:

1. A probe for an ultrasonic diagnostic apparatus, the probe comprising:
a transducer module including a transducer configured to transmit or receive ultrasound waves;
a first casing positioned below the transducer module to support the transducer module;
a second casing extending along a perimeter of the first casing, the second casing integrally formed with the first casing;
a heat spreader positioned below the transducer module and above the first casing, the heat spreader being a sheet of metal;
a printed circuit board (PCB) electrically connected to the transducer; and
a support frame configured to support the PCB, the support frame integrally formed with the first casing.

2. The probe of claim 1, wherein the first casing, the second casing, and/or the support frame is made of at least one material selected from polyphenylene sulfide (PPS), polyamide66+glass fiber (PA66+GF), polyphthalamide+GF (PPA+GF), terephthalic acid+GF (TPA+GF), polyetheretherketone+GF (PEEK+GF), PA66+long carbon fiber (PA66+LCF), PA66+long-glass fiber reinforced (PA66+LGF), polycarbonate+GF (PC+GF), PC+CF, PEEK, polyphenylsulfone (PPSU), polysulfone (PSU), PPS+GF+melamine formaldehyde resin (PPS+GF+MF), nylon-6+elastomer, and nylon-6+elastomer+GF.

3. The probe of claim 2, wherein the first casing, the second casing, and the support frame are integrally injection molded with one another.

4. The probe of claim 1, wherein the heat spreader has a thickness in a range of 3 mm to 10 mm.

5. The probe of claim 1, wherein the transducer module further comprises:
an acoustic reflective layer provided at a rear surface of the transducer to reflect ultrasound waves transmitted to a rear of the transducer; and
a backing layer provided at a rear surface of the acoustic reflective layer to prevent ultrasound waves from being transmitted to the rear of the transducer.

6. The probe of claim 1, further comprising a third casing fitted to the second casing and in a shape of a handle adapted to fit a user's hand.

7. The probe of claim 6, further comprising a heat sink in contact with the heat spreader and configured to receive heat from the heat spreader.

8. The probe of claim 7, wherein the heat sink comprises at least one heat sink plate having a planar shape.

9. The probe of claim 8, wherein the first casing comprises at least one heat sink through hole, wherein a portion of the at least one heat sink plate passes through the at least one heat sink through hole.

10. The probe of claim 8, wherein the at least one heat sink plate is made of graphite.

11. The probe of claim 7, further comprising a conductive oil accommodated in the second casing and having a higher thermal conductivity than air.

12. The probe of claim 11, wherein the conductive oil is in contact with a portion of the heat sink and is configured to transfer heat from the transducer to the heat sink.
